(19) United States
(12) Patent Application Publication
(10) Pub. No.: US 2020/0251214 A1
(43) Pub. Date: Aug. 6, 2020

(54) Liver Fibrosis Assessment Model, Liver Fibrosis Assessment System And Liver Fibrosis Assessment Method

(71) Applicant: China Medical University Hospital, Taichung City (TW)

(72) Inventors: Tzung-Chi Huang, Taichung City (TW); Ken Ying-Kai Liao, Taichung City (TW); Cheng-Yuan Peng, Taichung City (TW)

(73) Assignee: China Medical University Hospital, Taichung City (TW)

(21) Appl. No.: 16/701,506

(22) Filed: Dec. 3, 2019

(30) Foreign Application Priority Data
Feb. 1, 2019 (TW) 108104304

(51) Int. Cl. G16H 50/20; G16H 50/30; G16H 70/60; G16H 50/70; G16H 50/50; G16H 10/60; G01N 33/49; G06K 9/62

(52) U.S. Cl. CPC G16H 50/20; G16H 50/30; G16H 70/60; G16H 50/70; G16H 50/50; G01N 2800/52; G01N 33/49; G06K 9/6298; G06K 9/6217; G01N 2800/085; G01N 2800/7052; G16H 10/60

(57) ABSTRACT

A liver fibrosis assessment model includes following establishing steps. A reference database is obtained, wherein the reference database includes a plurality of reference blood test data. A preprocessing step of the blood test data is performed. A feature extracting step is performed, wherein the feature extracting step is for extracting at least one eigenvalue according to the reference database. A normalizing step of the blood test data is performed. A classifying step is performed, wherein the classifying step is for achieving a convergence of the normalized reference blood test data by using a gradient boosting algorithm so as to obtain the liver fibrosis assessment model. The liver fibrosis assessment model is used to assess whether a subject suffers from liver fibrosis and predict a degree of liver fibrosis of the subject.

### RELATED APPLICATIONS

This application claims priority to Taiwan Application Serial Number 108104304, filed Feb. 1, 2019, which is herein incorporated by reference.

### BACKGROUND

### Technical Field

The present disclosure relates to a medical information analysis model, system and method thereof. More particularly, the present disclosure relates to a liver fibrosis assessment model, a liver fibrosis assessment system and a liver fibrosis assessment method.

### Description of Related Art

Chronic hepatitis B and chronic hepatitis C are worldwide diseases and are also the major cause of liver cirrhosis and liver cancer (also known as hepatocellular carcinoma), and the degree of liver fibrosis is closely related to the development of liver cirrhosis. In detail, liver fibrosis may occur after the liver is inflamed and damaged, and the final outcome of liver fibrosis is cirrhosis. The patients with cirrhosis will have the opportunity to suffer from liver cancer. Accordingly, if the degree of liver fibrosis of subjects can be accurately and timely assessed so as to prevent it from evolving into cirrhosis, it is favorable for preventing the liver cancer greatly.

A liver biopsy, which removes a piece of tissue or a sample of liver from the subject's body directly, is the gold standard method for diagnosing the degree of liver fibrosis. However, the liver biopsy is an invasive medical test, and there is one-thousandth percentages of the subjects underwent the liver biopsy will face to the dangers of complications such as bleeding, infection, pneumothorax, and death. Thus, the willingness of patients to undergo liver biopsy is low.

Along with the advance of the imaging technology, non-invasive imaging testing methods start to apply to the diagnosis of liver fibrosis. The non-invasive imaging testing methods include conventional ultrasound imaging method, transient elastography (TE), ultrasound-based elastography and acoustic radiation force impulse (ARFI). In recent year, magnetic resonance elastography (MRE) is further applied to observe the image of the liver treated by magnetic fields and shock waves so as to calculate the degree of liver fibrosis according to the shear wave amplitude distribution of the image. However, the aforementioned imaging testing methods are not only time-consuming but also complicated in testing steps. Furthermore, the cost of the aforementioned imaging testing methods is expensive, making its clinical application less popular.

Therefore, how to develop a rapid, low-cost and highly accurate detecting method of liver fibrosis is a technical issue with clinical application value.

### SUMMARY

According to one aspect of the present disclosure, a liver fibrosis assessment model includes following establishing steps. A reference database is obtained, wherein the reference database includes a plurality of reference blood test data. A preprocessing step of the blood test data is performed, wherein the preprocessing step is for replacing a missing value of each of the reference blood test data with an average value of the reference blood test data. A feature extracting step is performed, wherein the feature extracting step is for extracting at least one eigenvalue according to the reference database. A normalizing step of the blood test data is performed, wherein a unit value of each of the reference blood test data is unified and then each of the reference blood test data is normalized by the at least one eigenvalue so as to obtain a plurality of normalized reference blood test data, and a value of each of the normalized reference blood test data ranges between −1 and 1. A classifying step is performed, wherein the classifying step is for achieving a convergence of the normalized reference blood test data by using a gradient boosting algorithm so as to obtain the liver fibrosis assessment model. The liver fibrosis assessment model is used to assess whether a subject suffers from liver fibrosis and predict a degree of liver fibrosis of the subject.

According to another aspect of the present disclosure, a liver fibrosis assessment system, which is for assessing whether a subject suffers from liver fibrosis and predicting a degree of liver fibrosis of the subject, includes a non-transitory machine readable medium. The non-transitory machine readable medium includes a storing unit and a processing unit, wherein the storing unit is for storing a target blood test data of the subject and a liver fibrosis assessment program, and the processing unit is for processing the liver fibrosis assessment program. The liver fibrosis assessment program includes a reference database storing module, a blood test data preprocessing module, a feature extracting module, a normalizing module, a liver fibrosis assessment model and a comparing module. The reference database storing module is for storing a reference database, wherein the reference database includes a plurality of reference blood test data. The blood test data preprocessing module is for replacing a missing value of each of the reference blood test data and a missing value of the target blood test data with an average value of the reference blood test data, respectively. The feature extracting module is for extracting at least one eigenvalue according to the reference database. The normalizing module is for unifying a unit value of each of the reference blood test data and a unit value of the target blood test data and then normalizing each of the reference blood test data and the target blood test data by the at least one eigenvalue so as to obtain a plurality of normalized reference blood test data and a normalized target blood test data, wherein a value of each of the normalized reference blood test data and the normalized target blood test data ranges between −1 and 1. The liver fibrosis assessment model establishing module is for achieving a convergence of the normalized reference blood test data by using a gradient boosting algorithm so as to obtain a liver fibrosis assessment model. The comparing module is for analyzing the normalized target blood test data by the liver fibrosis assessment model so as to obtain an eigenvalue weight data of liver fibrosis, wherein the eigenvalue weight data of liver fibrosis is used to assess whether the subject suffers from liver fibrosis and predict the degree of liver fibrosis of the subject.

According to another aspect of the present disclosure, a liver fibrosis assessment method includes following steps. The liver fibrosis assessment model according to the aforementioned aspect is provided. A target blood test data of the subject is provided. The target blood test data is preprocessed, wherein a missing value of the target blood test data is replaced with the average value of the reference blood test data. The target blood test data is normalized, wherein a unit value of the target blood test data is unified with the unit value of each of the reference blood test data and then the target blood test data is normalized by the at least one eigenvalue so as to obtain a normalized target blood test data, and a value of the normalized target blood test data ranges between −1 and 1. The normalized target blood test data is analyzed by the liver fibrosis assessment model so as to assess whether the subject suffers from liver fibrosis and predict the degree of liver fibrosis of the subject.

### BRIEF DESCRIPTION OF THE DRAWINGS

The present disclosure can be more fully understood by reading the following detailed description of the embodiment, with reference made to the accompanying drawings as follows:

FIG. 1 is a flow chart of establishing steps of a liver fibrosis assessment model according to one embodiment of the present disclosure.

FIG. 2 is a block diagram of a liver fibrosis assessment system according to another embodiment of the present disclosure.

FIG. 3 is a flow chart of a liver fibrosis assessment method according to further another embodiment of the present disclosure.

FIG. 4 is a receiver operating characteristic curve (ROC) diagram of the liver fibrosis assessment model of the present disclosure.

FIG. 5 is one confusion matrix diagram of the liver fibrosis assessment model of the present disclosure used to assess a degree of liver fibrosis of subjects.

FIG. 6 is another confusion matrix diagram of the liver fibrosis assessment model of the present disclosure used to assess the degree of liver fibrosis of subjects.

FIG. 7 is further another confusion matrix diagram of the liver fibrosis assessment model of the present disclosure used to assess the degree of liver fibrosis of subjects.

### DETAILED DESCRIPTION

The present disclosure will be further exemplified by the following specific embodiments so as to facilitate utilizing and practicing the present disclosure completely by the people skilled in the art without over-interpreting and over-experimenting. However, these practical details are used to describe how to implement the materials and methods of the present disclosure and are not necessary.

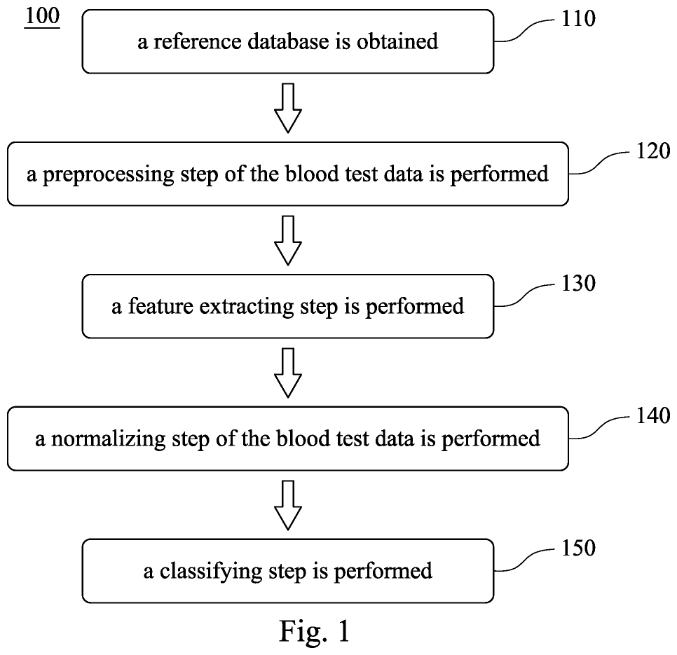

Please refer to FIG. 1, which is a flow chart of establishing steps of a liver fibrosis assessment model 100 according to one embodiment of the present disclosure. The establishing steps of the liver fibrosis assessment model 100 include Step 110, Step 120, Step 140 and Step 150.

In Step 110, a reference database is obtained, wherein the reference database includes a plurality of reference blood test data. In detail, the reference blood test data are indirect marker data of serum test from the blood sample of subjects, and the liver fibrosis assessment model of the present disclosure uses the aforementioned indirect marker data of serum test for analysis. Accordingly, it is favorable for avoiding the complications caused by the invasive medical test of liver fibrosis, and the reference blood test data can be used directly to assess a degree of liver fibrosis of subjects. More preferably, the reference blood test data can include a reference subject physiological age data, a reference aspartate aminotransferase (AST) index, a reference alanine aminotransferase (ALT) index and a reference platelet count data. The reference subject physiological age data, the reference AST index, the reference ALT index and the reference platelet count data are called “Fibrosis 4 score (FIB-4 score)”, wherein a ratio of the reference AST index and the reference platelet count data is called “Aspartate aminotransferase to platelet ratio index (APRI)”.

FIB-4 score is the current assessing basis of serum test of liver fibrosis, and APRI is used for predicting the degree of liver fibrosis or cirrhosis of the patients suffered from chronic hepatitis B and chronic hepatitis C. In detail, when the FIB-4 score of a patient is larger than 3.25, the patient can be classified into a patient with advanced liver fibrosis, and the accuracy thereof is up to 97%. Furthermore, an accuracy of the positive predictive value of patients with HIV/HCV co-infection by FIB-4 score can also be more than 65%.

In Step 120, a preprocessing step of the blood test data is performed, wherein the preprocessing step is for replacing a missing value of each of the reference blood test data with an average value of the reference blood test data. In detail, in order to prevent misdiagnosis of the liver fibrosis assessment model caused by a null value of the reference blood test data, such as missing of the test data, so as to enhance the assessing accuracy thereof, the missing value of each of the reference blood test data will be replaced with the average value of the reference blood test data so as to reduce the duplicate rate of the data effectively.

More preferably, the preprocessing step of the blood test data can calculate an average value of the reference subject physiological age data of the reference blood test data, an average value of the reference AST indexes of the reference blood test data, an average value of the reference ALT indexes of the reference blood test data and an average value of the reference platelet count data of the reference blood test data, respectively. Then, the preprocessing step of the blood test data can further replace a missing value of the reference subject physiological age data with the average value of the reference subject physiological age data of the reference blood test data, replace a missing value of the reference AST indexes with the average value of the reference AST indexes of the reference blood test data, replace a missing value of the reference ALT indexes with the average value of the reference ALT indexes of the reference blood test data and replace a missing value of the reference platelet count data with the average value of the reference platelet count data of the reference blood test data. Therefore, it is favorable for enhancing the assessing accuracy of liver fibrosis of the liver fibrosis assessment model of the present disclosure.

In Step 130, a feature extracting step is performed, wherein the feature extracting step is for extracting at least one eigenvalue according to the reference database. In detail, in the feature extracting step, the at least one eigenvalue according to the reference database can be automatically extracted by filter method, wrapper method or embedded method so as to confirm the characteristic value of the reference database. Furthermore, in the feature extracting step, at least one eigenvalue according to the reference subject physiological age data, at least one eigenvalue according to the reference AST indexes, at least one eigenvalue according to the reference ALT indexes and at least one eigenvalue according to the reference platelet count data will be extracted, respectively, so as to facilitate the following establishing steps.

In Step 140, a normalizing step of the blood test data is performed, wherein a unit value of each of the reference blood test data is unified and then each of the reference blood test data is normalized by the at least one eigenvalue so as to obtain a plurality of normalized reference blood test data, and a value of each of the normalized reference blood test data ranges between −1 and 1. In detail, in the reference blood test data of the present disclosure, the unit values of the reference subject physiological age data thereof, the unit values of the reference AST indexes thereof, the unit values of the reference ALT indexes thereof and the unit values of the reference platelet count data thereof may be different, so that the normalizing step of the blood test data can change the unit values of the reference subject physiological age data to the same, change the unit values of the reference AST index to the same, change the unit values of the reference ALT index to the same and change the unit values of the reference platelet count data to the same. Therefore, the liver fibrosis assessment model of the present disclosure can have the same weight standard in each of the reference blood test data. Furthermore, in the normalizing step of the blood test data, each of the reference subject physiological age data, each of the reference AST index, each of the reference ALT index and each of the reference platelet count data will be normalized according to the at least one eigenvalue of the reference subject physiological age data, the at least one eigenvalue of the reference AST indexes, the at least one eigenvalue of the reference ALT indexes and the at least one eigenvalue of the reference platelet count data, respectively, by Formula I so as to obtain a plurality of normalized reference subject physiological age data, a plurality of normalized reference AST indexes, a plurality of normalized reference ALT indexes, a plurality of normalized reference platelet count data, and Formula I for normalized the reference blood test data is shown as below:

Normalized value z=(x−u)+s (Formula I),

wherein x is the eigenvalue according to the reference subject physiological age data, the reference AST indexes, the reference ALT indexes or the reference platelet count data of the reference blood test data, u is the average value of the reference subject physiological age data, the reference AST indexes, the reference ALT indexes or the reference platelet count data of the reference blood test data, and s is the standard deviation of the reference subject physiological age data, the reference AST indexes, the reference ALT indexes or the reference platelet count data of the reference blood test data. After the normalizing step of the blood test data is performed, a value of each of the normalized reference subject physiological age data, the normalized reference AST indexes, the normalized reference ALT indexes or the normalized reference platelet count data will range between −1 and 1, so that the assessing speed of the liver fibrosis assessment model of the present disclosure can be further enhanced. Therefore, the assessing accuracy of liver fibrosis of the liver fibrosis assessment model of the present disclosure can be enhanced, and it is favorable for improving the classifying efficiency of the gradient boosting algorithm as follows.

In Step 150, a classifying step is performed, wherein the classifying step is for achieving a convergence of the normalized reference blood test data by using a gradient boosting algorithm so as to obtain the liver fibrosis assessment model of the present disclosure, and the liver fibrosis assessment model of the present disclosure is used to assess whether a subject suffers from liver fibrosis and predict the degree of liver fibrosis of the subject. Therefore, it is favorable for enhancing the assessing accuracy of the liver fibrosis assessment model of the present disclosure and preventing the prediction difference of the reference blood test data assessed by the liver fibrosis assessment model thereof from being too high or too low.

More preferably, the degree of liver fibrosis of the subject can be mild liver fibrosis, moderate liver fibrosis, serious liver fibrosis or severe liver fibrosis. In detail, METAVIR scoring system is a system used to assess the extent of inflammation and fibrosis by histopathological evaluation in a liver biopsy of patients and has five fibrosis stages being F0 to F4, wherein the stage F0 means the patient has no symptom of liver fibrosis, the stage F1 means the liver of the patient has portal fibrosis without septa, which belongs to mild liver fibrosis, the stage F2 means the liver of the patient has portal fibrosis with few septa, which belongs to moderate liver fibrosis, the stage F3 means the liver of the patient has numerous septa without cirrhosis, which belongs to serious liver fibrosis, and stage F4 means the degree of liver fibrosis of the patient is severe liver fibrosis and can be directly classified into a patient with cirrhosis. Therefore, the liver fibrosis assessment model of the present disclosure can classify and train the reference blood test data thereof by the gradient boosting algorithm, and the predicted degree of liver fibrosis assessed by the liver fibrosis assessment model of the present disclosure can be consistent with the liver fibrosis grading result of the histopathological liver fibrosis evaluation method, so that the liver fibrosis assessment model of the present disclosure has excellent evaluation accuracy and has the potential applied in the clinically related field.

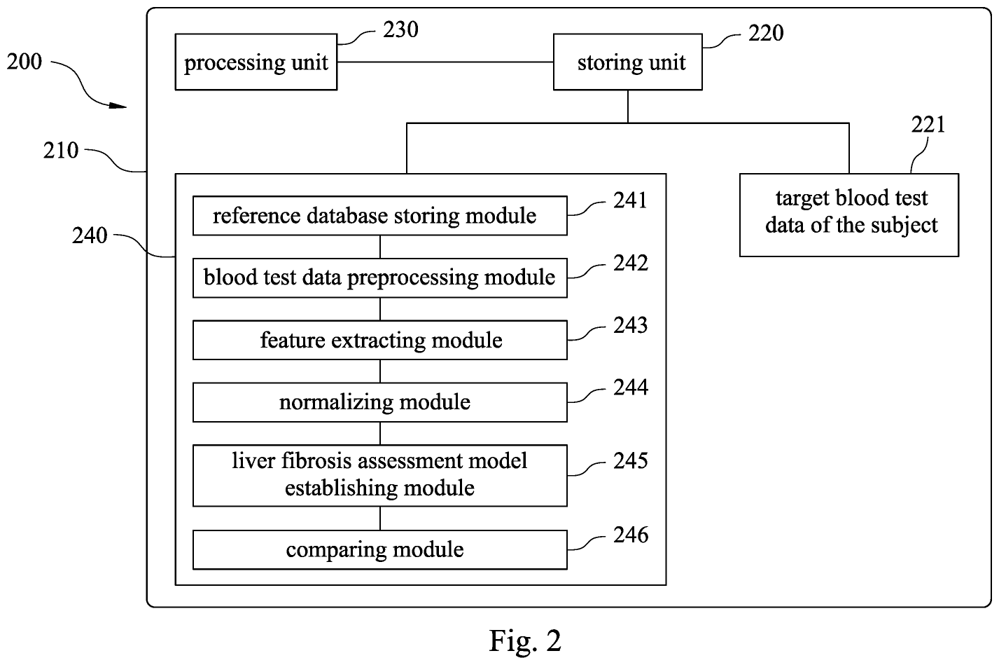

Please refer to FIG. 2, which is a block diagram of a liver fibrosis assessment system 200 according to another embodiment of the present disclosure. The liver fibrosis assessment system 200 is for assessing whether the subject suffers from liver fibrosis and predicting the degree of liver fibrosis of the subject, and the liver fibrosis assessment system 200 includes a non-transitory machine readable medium 210. The non-transitory machine readable medium 210 includes a storing unit 220 and a processing unit 230, wherein the storing unit 220 is for storing a target blood test data 221 of the subject and a liver fibrosis assessment program 240, and the processing unit 230 is for processing the liver fibrosis assessment program 240. The liver fibrosis assessment program 240 includes a reference database storing module 241, a blood test data preprocessing module 242, a feature extracting module 243, a normalizing module 244, a liver fibrosis assessment model establishing module 245 and a comparing module 246. More preferably, the target blood test data 221 can include a target subject physiological age data, a target AST index, a target ALT index and a target platelet count data.

The reference database storing module 241 is for storing a reference database, wherein the reference database includes a plurality of reference blood test data. More preferably, each of the reference blood test data can include a reference subject physiological age data, a reference AST index, a reference ALT index and a reference platelet count data. Therefore, it is favorable for the liver fibrosis assessment program 240 to assess the degree of liver fibrosis of the subject more accurately, and the reference blood test data can be used directly to assess the degree of liver fibrosis of subjects by the liver fibrosis assessment system 200 of the present disclosure.

The blood test data preprocessing module 242 is for replacing a missing value of each of the reference blood test data and a missing value of the target blood test data 221 with an average value of the reference blood test data, respectively. More preferably, the blood test data preprocessing module 242 can be used for calculating an average value of the reference subject physiological age data of the reference blood test data, an average value of the reference AST indexes of the reference blood test data, an average value of the reference ALT indexes of the reference blood test data and an average value of the reference platelet count data of the reference blood test data, respectively, and then replacing a missing value of the reference subject physiological age data with the average value of the reference subject physiological age data of the reference blood test data, replacing a missing value of the reference AST indexes with the average value of the reference AST indexes of the reference blood test data, replacing a missing value of the reference ALT indexes with the average value of the reference ALT indexes of the reference blood test data and replacing a missing value of the reference platelet count data with the average value of the reference platelet count data of the reference blood test data. Therefore, it is favorable for enhancing the assessing accuracy of the liver fibrosis of the liver fibrosis assessment system 200 of the present application.

More, preferably, the blood test data preprocessing module 242 can be used for replacing a missing value of the target subject physiological age data with the average value of the reference subject physiological age data of the reference blood test data, replacing a missing value of the target AST indexes with the average value of the reference AST indexes of the reference blood test data, replacing a missing value of the target ALT indexes with the average value of the reference ALT indexes of the reference blood test data and replacing a missing value of the target platelet count data with the average value of the reference platelet count data of the reference blood test data.

The feature extracting module 243 is for extracting at least one eigenvalue according to the reference database. In detail, the at least one eigenvalue can be automatically extracted by the feature extracting module 243 using filter method, wrapper method or embedded method. More preferably, at least one eigenvalue of the reference subject physiological age data, at least one eigenvalue of the reference AST indexes, at least one eigenvalue of the reference ALT indexes and at least one eigenvalue of the reference platelet count data can be extracted by the feature extracting module 243, respectively.

The normalizing module 244 is for unifying a unit value of each of the reference blood test data and a unit value of the target blood test data 221 and then normalizing each of the reference blood test data and the target blood test data 221 by the at least one eigenvalue so as to obtain a plurality of normalized reference blood test data and a normalized target blood test data, wherein a value of each of the normalized reference blood test data and the normalized target blood test data ranges between −1 and 1. More preferably, the normalizing module 244 can change the unit values of the reference subject physiological age data to the same, change the unit values of the reference AST indexes to the same, change the unit values of the reference ALT indexes to the same and change the unit values of the reference platelet count data to the same. After that, the unit value of the target subject physiological age data, the unit value of the target AST index, the unit value of the target ALT index and the unit value of the target platelet count data can be further changed to be identical with the unit of the reference subject physiological age data, the unit value of the reference AST indexes, the unit value of the reference ALT indexes and the unit value of the reference platelet count data, respectively, by the normalizing module 244. After the unit values of the reference blood test data and the unit value of the target blood test data 221 are unified, each of the reference subject physiological age data, each of the reference AST indexes, each of the reference ALT indexes, each of the reference platelet count data, the target subject physiological age data, the target AST index, the target ALT index and the target platelet count data will be normalized according to the at least one eigenvalue of the reference subject physiological age data, at least one eigenvalue of the reference AST indexes, at least one eigenvalue of the reference ALT indexes and at least one eigenvalue of the reference platelet count data, respectively, by the normalizing module 244 according to Formula I. Thus, a plurality of normalized reference subject physiological age data, a plurality of normalized reference AST indexes, a plurality of normalized reference ALT indexes, a plurality of normalized reference platelet count data, a normalized target subject physiological age data, a normalized target AST index, a normalized target ALT index and a normalized target platelet count data ranging between −1 and 1 can be obtained. Therefore, the assessing speed of the liver fibrosis assessment model of the present disclosure can be further enhanced and the assessing accuracy thereof can also be further enhanced.

The liver fibrosis assessment model establishing module 245 is for achieving a convergence of the normalized reference blood test data by using a gradient boosting algorithm so as to obtain the liver fibrosis assessment model of the present disclosure.

The comparing module 246 is for analyzing the normalized target blood test data by the liver fibrosis assessment model so as to obtain an eigenvalue weight data of liver fibrosis, wherein the eigenvalue weight data of liver fibrosis is used to assess whether the subject suffers from liver fibrosis and predict the degree of liver fibrosis of the subject.

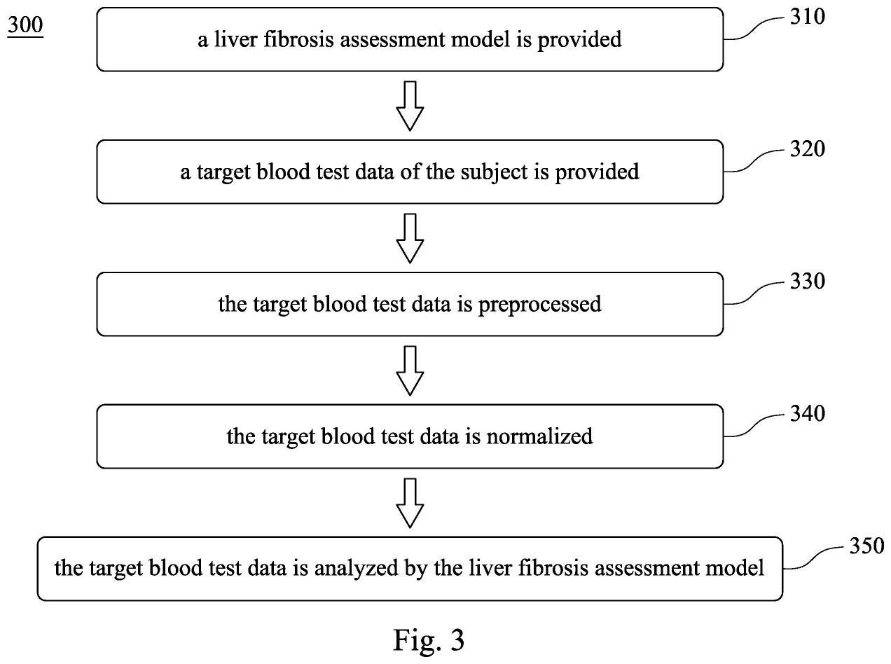

Please refer to FIG. 3, which is a flow chart of a liver fibrosis assessment method 300 according to further another embodiment of the present disclosure. The liver fibrosis assessment method 300 includes Step 310, Step 320, Step 330, Step 340 and Step 350.

In Step 310, a liver fibrosis assessment model is provided. In detail, the aforementioned liver fibrosis assessment model is established by Step 110 to Step 150 as the foregoing description.

In Step 320, a target blood test data of the subject is provided. More preferably, the target blood test data can include a target subject physiological age data, a target AST index, a target ALT index and a target platelet count data.

In Step 330, the target blood test data is preprocessed, wherein a missing value of the target blood test data is replaced with the average value of the reference blood test data described in Step 120. More preferably, each of the reference blood test data can include a reference subject physiological age data, a reference AST index, a reference ALT index and a reference platelet count data so as to preprocess the target subject physiological age data, the target AST index, the target ALT index and the target platelet count data of the target blood test data more accurately.

In Step 340, the target blood test data is normalized, wherein a unit value of the target blood test data is unified with the unit value of each of the reference blood test data and then the target blood test data is normalized by the at least one eigenvalue so as to obtain a normalized target blood test data. A value of the normalized target blood test data ranges between −1 and 1 so as to enhance the assessing speed of the liver fibrosis assessment model.

In Step 350, the target blood test data is analyzed by the liver fibrosis assessment model so as to assess whether the subject suffers from liver fibrosis and predict the degree of liver fibrosis of the subject. More preferably, the degree of liver fibrosis can be mild liver fibrosis, moderate liver fibrosis, serious liver fibrosis or severe liver fibrosis.

The present disclosure will be further exemplified by the following specific examples according to the aforementioned description.

### Examples

### I. Reference Database

The reference database used in the present disclosure is the retrospective clinical blood test data of subjects collected by China Medical University Hospital. This clinical trial program is approved by China Medical University & Hospital Research Ethics Committee, which is numbered as CMUH 107-REC1-129.

The reference database includes 2354 de-linked reference blood test data of subjects, wherein each of the de-linked reference blood test data includes a subject physiological age data, an AST index, an ALT index and a platelet count data so as to meet the current criteria of blood tests for liver fibrosis.

### II. The Liver Fibrosis Assessment Model of the Present Disclosure

After the reference database is obtained, the liver fibrosis assessment model of the present disclosure will calculate an average value of the reference subject physiological age data of the reference blood test data, an average value of the reference AST indexes of the reference blood test data, an average value of the reference ALT indexes of the reference blood test data and an average value of the reference platelet count data of the reference blood test data, respectively. Then, the liver fibrosis assessment model of the present disclosure can further replace a missing value of the reference subject physiological age data with the average value of the reference subject physiological age data of the reference blood test data, replace a missing value of the reference AST indexes with the average value of the reference AST indexes of the reference blood test data, replace a missing value of the reference ALT indexes with the average value of the reference ALT indexes of the reference blood test data and replace a missing value of the reference platelet count data with the average value of the reference platelet count data of the reference blood test data.

Next, the unit values of the reference subject physiological age data, the unit values of the reference AST indexes, the unit values of the reference ALT indexes and the unit values of the reference platelet count data of the reference blood test data will be respectively normalized. Then, each of the reference subject physiological age data, each of the reference AST index, each of the reference ALT index and each of the reference platelet count data will be normalized according to at least one eigenvalue of the reference subject physiological age data, at least one eigenvalue of the reference AST indexes, at least one eigenvalue of the reference ALT indexes and at least one eigenvalue of the reference platelet count data, respectively, so as to obtain normalized reference blood test data include a plurality of normalized reference subject physiological age data, a plurality of normalized reference AST indexes, a plurality of normalized reference ALT indexes, a plurality of normalized reference platelet count data.

Next, a convergence of the normalized reference blood test data will be achieved by gradient boosting algorithm so as to obtain the liver fibrosis assessment model of the present disclosure. In detail, the gradient boosting algorithm uses a gradient descent algorithm and a boosting algorithm to analyze the normalized reference blood test data. Specifically, when the normalized reference blood test data are trained and classified and then achieved a convergence by one of the gradient descent algorithm and the boosting algorithm, in order to prevent the prediction difference of the normalized reference blood test data assessed by the liver fibrosis assessment model of the present disclosure from being too high or too low, the other of the gradient descent algorithm and the boosting algorithm of the gradient boosting algorithm will further be used so as to train and classify the aforementioned results (that is, the convergence achieved by the one of the gradient descent algorithm and the boosting algorithm). Therefore, it is favorable for ensuring that the loss function thereof can reach stable convergence.

### III. The Liver Fibrosis Assessment Model of the Present Disclosure Use to Assess the Whether a Subject Suffers from Liver Fibrosis and Predict the Degree of Liver Fibrosis of the Subject

In the present experiment, the liver fibrosis assessment model established by the aforementioned steps will be used to assess that whether the subject suffers from liver fibrosis and predict the degree of liver fibrosis of the subject, and the assessing steps are processed sequentially as follow. First, the liver fibrosis assessment model established by the aforementioned steps is provided. Next, the target blood test data of the subject is provided. Then, the target blood test data is preprocessed, wherein a missing value of the target blood test data is replaced with the average value of the reference blood test data. Then, the target blood test data is normalized, wherein a unit value of the target blood test data is unified with the unit value of each of the reference blood test data and then the target blood test data is normalized by the at least one eigenvalue so as to obtain a normalized target blood test data, and a value of the normalized target blood test data ranges between −1 and 1. Finally, the normalized target blood test data is analyzed by the liver fibrosis assessment model so as to assess whether the subject suffers from liver fibrosis and predict the degree of liver fibrosis of the subject.

Next, the assessed results of whether the subject suffers from liver fibrosis and the degree of liver fibrosis of the subject will further be integrated to the reference database so as to optimize the liver fibrosis assessment model of the present disclosure. Therefore, the classifying effectivity and the assessing accuracy of the liver fibrosis assessment model can be further enhanced.

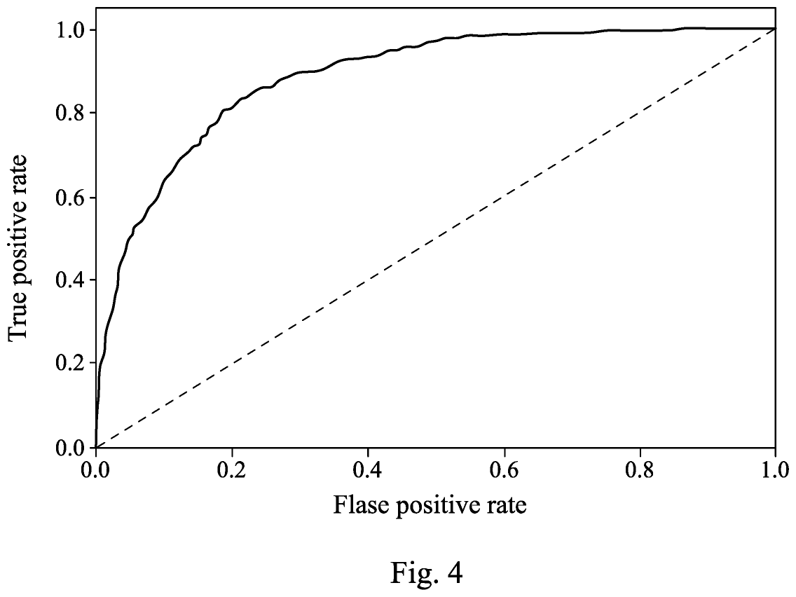

Please refer to FIG. 4, which is a receiver operating characteristic curve (ROC) diagram of the liver fibrosis assessment model of the present disclosure. As shown in FIG. 4, when the liver fibrosis assessment model of the present disclosure is used to assess whether the subject suffers from liver fibrosis, the area under the receiver operating characteristic curve (AUROC) thereof can reach to 88.4%. Furthermore, according to the current clinical data, the area under the receiver operating characteristic curve of the assessment of FIB-4 score used to assess the degree of liver fibrosis and the assessing sensitivity of the deterioration of cirrhosis in patients with chronic hepatitis B or C is 82%, and the area under the receiver operating characteristic curve of the assessing accuracy thereof is 86%. Therefore, the liver fibrosis assessment model, the liver fibrosis assessment system and the liver fibrosis assessment method of the present disclosure can be used to assess whether the subject suffers from liver fibrosis and the degree of liver fibrosis of the subject effectively and has the potential applied in the clinically related field.

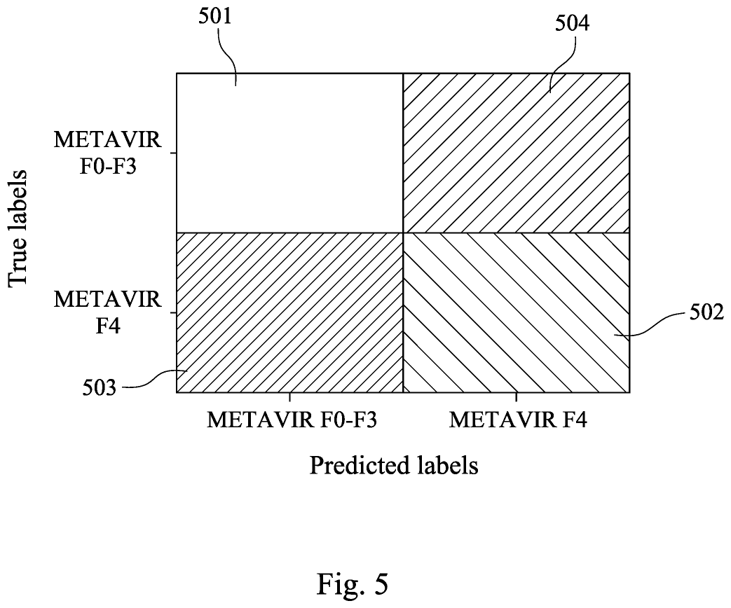

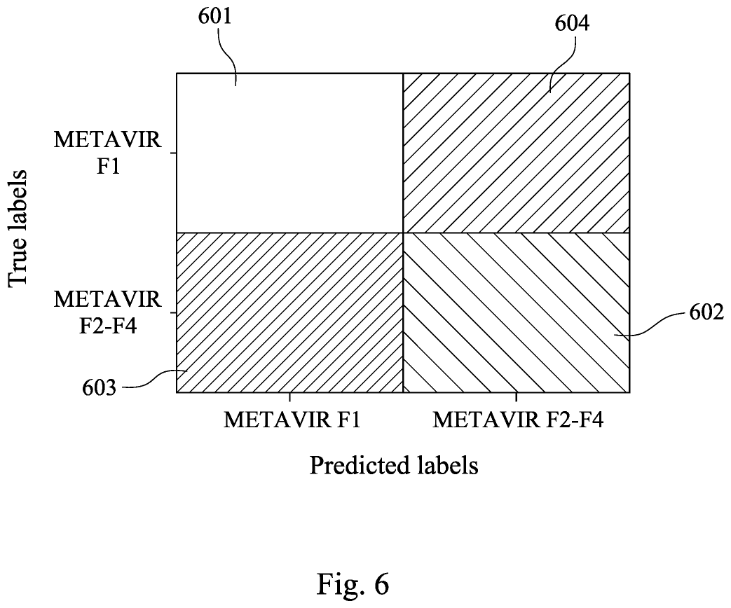

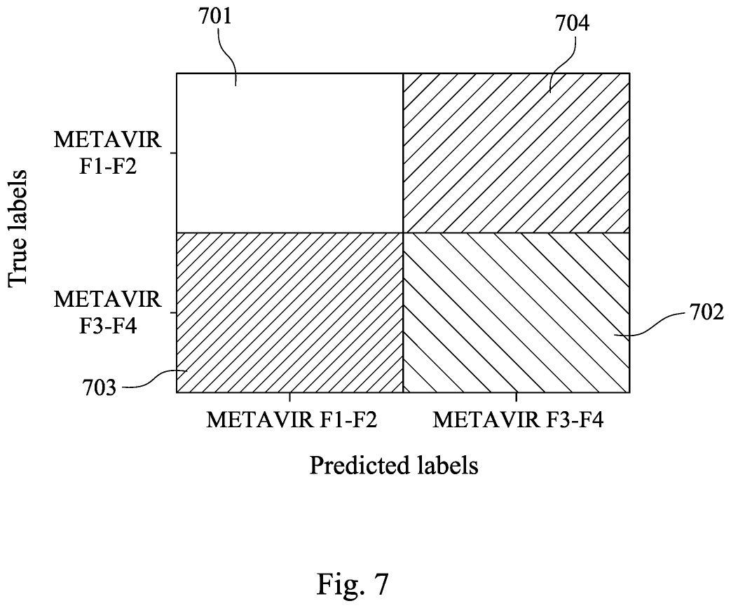

Please refer to FIG. 5, FIG. 6 and FIG. 7, wherein FIG. 5 is one confusion matrix diagram of the liver fibrosis assessment model of the present disclosure used to assess the degree of liver fibrosis of subjects, FIG. 6 is another confusion matrix diagram of the liver fibrosis assessment model of the present disclosure used to assess the degree of liver fibrosis of subjects, and FIG. 7 is further another confusion matrix diagram of the liver fibrosis assessment model of the present disclosure used to assess the degree of liver fibrosis of subjects. When the liver fibrosis assessment model of the present disclosure is used to assess the degree of liver fibrosis of the subject, the fibrosis stages of METAVIR scoring system are used as the standard to process the analysis, wherein the stages F0-F3 mean the liver of the subject is without cirrhosis, and the stage F4 means the degree of liver fibrosis of the subject is severe liver fibrosis, that is, the subject suffers from liver cirrhosis. In detail, FIG. 5 is a confusion matrix diagram showing the results of the liver fibrosis assessment model of the present disclosure used to assess the liver cirrhosis (F4 in METAVIR scoring system), FIG. 6 and FIG. 7 are confusion matrix diagrams showing the results of the liver fibrosis assessment model of the present disclosure used to assess the degree of liver fibrosis of subjects (F1 vs F2-F4 in FIG. 6 and F1-F2 vs F3-F4 in FIG. 7).

As shown in FIG. 5, the number of the subjects falling in the true negative (TN) block 501, which is without liver cirrhosis, of the predicted labels is 1609, the number of the subjects falling in the true positive (TP) block 502, which is with liver cirrhosis, of the predicted labels is 329, the number of the subjects falling in the false negative (FN) block 503, which is without liver cirrhosis, of the predicted labels is 196, and the number of the subjects falling in the false positive (FP) block 504, which is with liver cirrhosis, of the predicted labels is 206. As shown in FIG. 6, the number of the subjects falling in the true negative (TN) block 601 of the predicted labels is 360, the number of the subjects falling in the true positive (TP) block 602 of the predicted labels is 1388, the number of the subjects falling in the false negative (FN) block 603 of the predicted labels is 305, and the number of the subjects falling in the false positive (FP) block 604 of the predicted labels is 287. As shown in FIG. 7, the number of the subjects falling in the true negative (TN) block 701 of the predicted labels is 1303, the number of the subjects falling in the true positive (TP) block 702 of the predicted labels is 544, the number of the subjects falling in the false negative (FN) block 703 of the predicted labels is 341, and the number of the subjects falling in the false positive (FP) block 704 of the predicted labels is 152.

Furthermore, please refer to FIG. 5, FIG. 6, FIG. 7 and Table 1 simultaneously, wherein Table 1 shows the assessing results of the liver fibrosis assessment model of the present disclosure used to assess the degree of liver fibrosis of the subjects.

TABLE 1

| | | |
|---|---|---|
| F1-F3 (Negative) | Accuracy | 82.82% |
| vs | Sensitivity | 63% |
| F4 (Positive) | Specificity | 89% |
| | Positive Predictive Value | 61% |
| | Negative Predictive Value | 89% |
| F1-F2 (Negative) | Accuracy | 78.93% |
| vs | Sensitivity | 61% |
| F3~4 (Positive) | Specificity | 90% |
| | Positive Predictive Value | 78% |
| | Negative Predictive Value | 79% |
| F1 (Negative) | Accuracy | 74.7% |
| vs | Sensitivity | 56% |
| F2-F4 (Positive) | Specificity | 82% |
| | Positive Predictive Value | 54% |
| | Negative Predictive Value | 83% |

As shown in the aforementioned results, all of the accuracy, the sensitivity and the specificity of the liver fibrosis assessment model of the present disclosure are excellent. Thus, the liver fibrosis assessment model, the liver fibrosis assessment system and the liver fibrosis assessment method of the present disclosure can be used to assess the degree of liver fibrosis of the subjects according to target blood test data correctly.

To sum up, the reference blood test data and the target blood test data can be preprocessed and normalized by the liver fibrosis assessment model, the liver fibrosis assessment system and the liver fibrosis assessment method of the present disclosure and then trained by the gradient boosting algorithm so as to assess whether a subject suffers from liver fibrosis and predict a degree of liver fibrosis of the subject automatically according to the conventional blood test data. Therefore, it is favorable for avoiding the risks of the liver biopsy and greatly enhancing the assessing effectivity of liver fibrosis.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A liver fibrosis assessment model, comprising following establishing steps:
obtaining a reference database, wherein the reference database comprises a plurality of reference blood test data;
performing a preprocessing step of the blood test data, wherein the preprocessing step is for replacing a missing value of each of the reference blood test data with an average value of the reference blood test data;
performing a feature extracting step, wherein the feature extracting step is for extracting at least one eigenvalue according to the reference database;
performing a normalizing step of the blood test data, wherein a unit value of each of the reference blood test data is unified and then each of the reference blood test data is normalized by the at least one eigenvalue so as to obtain a plurality of normalized reference blood test data, and a value of each of the normalized reference blood test data ranges between −1 and 1;
performing a classifying step, wherein the classifying step is for achieving a convergence of the normalized reference blood test data by using a gradient boosting algorithm so as to obtain the liver fibrosis assessment model;
wherein the liver fibrosis assessment model is used to assess whether a subject suffers from liver fibrosis and predict a degree of liver fibrosis of the subject.

2. The liver fibrosis assessment model of claim 1, wherein each of the reference blood test data comprises a reference subject physiological age data, a reference aspartate aminotransferase (AST) index, a reference alanine aminotransferase (ALT) index and a reference platelet count data.

3. The liver fibrosis assessment model of claim 2, wherein the preprocessing step of the blood test data is for calculating an average value of the reference subject physiological age data of the reference blood test data, an average value of the reference AST indexes of the reference blood test data, an average value of the reference ALT indexes of the reference blood test data and an average value of the reference platelet count data of the reference blood test data, respectively, and then replacing a missing value of the reference subject physiological age data with the average value of the reference subject physiological age data of the reference blood test data, replacing a missing value of the reference AST indexes with the average value of the reference AST indexes of the reference blood test data, replacing a missing value of the reference ALT indexes with the average value of the reference ALT indexes of the reference blood test data and replacing a missing value of the reference platelet count data with the average value of the reference platelet count data of the reference blood test data.

4. The liver fibrosis assessment model of claim 1, wherein the degree of liver fibrosis of the subject is mild liver fibrosis, moderate liver fibrosis, serious liver fibrosis or severe liver fibrosis.

5. A liver fibrosis assessment system, which is for assessing whether a subject suffers from liver fibrosis and predicting a degree of liver fibrosis of the subject, comprising:
a non-transitory machine readable medium comprising a storing unit and a processing unit, wherein the storing unit is for storing a target blood test data of the subject and a liver fibrosis assessment program, and the processing unit is for processing the liver fibrosis assessment program;
wherein the liver fibrosis assessment program comprises:
a reference database storing module for storing a reference database, wherein the reference database comprises a plurality of reference blood test data;
a blood test data preprocessing module for replacing a missing value of each of the reference blood test data and a missing value of the target blood test data with an average value of the reference blood test data, respectively;
a feature extracting module for extracting at least one eigenvalue according to the reference database;
a normalizing module for unifying a unit value of each of the reference blood test data and a unit value of the target blood test data and then normalizing each of the reference blood test data and the target blood test data by the at least one eigenvalue so as to obtain a plurality of normalized reference blood test data and a normalized target blood test data, wherein a value of each of the normalized reference blood test data and the normalized target blood test data ranges between −1 and 1;
a liver fibrosis assessment model establishing module for achieving a convergence of the normalized reference blood test data by using a gradient boosting algorithm so as to obtain a liver fibrosis assessment model; and
a comparing module for analyzing the normalized target blood test data by the liver fibrosis assessment model so as to obtain an eigenvalue weight data of liver fibrosis, wherein the eigenvalue weight data of liver fibrosis is used to assess whether the subject suffers from liver fibrosis and predict the degree of liver fibrosis of the subject.

6. The liver fibrosis assessment system of claim 5, wherein each of the reference blood test data comprises a reference subject physiological age data, a reference AST index, a reference ALT index and a reference platelet count data, and the target blood test data comprises a target subject physiological age data, a target AST index, a target ALT index and a target platelet count data.

7. The liver fibrosis assessment system of claim 6, wherein the blood test data preprocessing module is for calculating an average value of the reference subject physiological age data of the reference blood test data, an average value of the reference AST indexes of the reference blood test data, an average value of the reference ALT indexes of the reference blood test data and an average value of the reference platelet count data of the reference blood test data, respectively, and then replacing a missing value of the reference subject physiological age data with the average value of the reference subject physiological age data of the reference blood test data, replacing a missing value of the reference AST indexes with the average value of the reference AST indexes of the reference blood test data, replacing a missing value of the reference ALT indexes with the average value of the reference ALT indexes of the reference blood test data and replacing a missing value of the reference platelet count data with the average value of the reference platelet count data of the reference blood test data.

8. The liver fibrosis assessment system of claim 7, wherein the blood test data preprocessing module is for replacing a missing value of the target subject physiological age data with the average value of the reference subject physiological age data of the reference blood test data, replacing a missing value of the target AST indexes with the average value of the reference AST indexes of the reference blood test data, replacing a missing value of the target ALT indexes with the average value of the reference ALT indexes of the reference blood test data and replacing a missing value of the target platelet count data with the average value of the reference platelet count data of the reference blood test data.

9. The liver fibrosis assessment system of claim 5, wherein the degree of liver fibrosis of the subject is mild liver fibrosis, moderate liver fibrosis, serious liver fibrosis or severe liver fibrosis.

10. A liver fibrosis assessment method, comprising:
providing the liver fibrosis assessment model of claim 1;
providing a target blood test data of the subject;
preprocessing the target blood test data, wherein a missing value of the target blood test data is replaced with the average value of the reference blood test data;
normalizing the target blood test data, wherein a unit value of the target blood test data is unified with the unit value of each of the reference blood test data and then the target blood test data is normalized by the at least one eigenvalue so as to obtain a normalized target blood test data, and a value of the normalized target blood test data ranges between −1 and 1; and
analyzing the normalized target blood test data by the liver fibrosis assessment model so as to assess whether the subject suffers from liver fibrosis and predict the degree of liver fibrosis of the subject.

11. The liver fibrosis assessment method of claim 10, wherein each of the reference blood test data comprises a reference subject physiological age data, a reference AST index, a reference ALT index and a reference platelet count data, and the target blood test data comprises a target subject physiological age data, a target AST index, a target ALT index and a target platelet count data.

12. The liver fibrosis assessment method of claim 10, wherein the degree of liver fibrosis of the subject is mild liver fibrosis, moderate liver fibrosis, serious liver fibrosis or severe liver fibrosis.
